(19) United States
(12) Patent Application Publication
(10) Pub. No.: US 2007/0172021 A1
(43) Pub. Date: Jul. 26, 2007

(54) Method for imaging in a medical diagnostic unit, and diagnostic unit therefor

(76) Inventor: Michael Deimling, Mohrendorf (DE)

Correspondence Address: HARNESS, DICKEY & PIERCE, P.L.C., P.O.BOX 8910, RESTON, VA, 20195, US

(21) Appl. No.: 11/648,670

(22) Filed: Jan. 3, 2007

(30) Foreign Application Priority Data
Jan. 4, 2006 (DE) 10 2006 000 789.1

(51) Int. Cl. H05G 1/60; A61B 6/00; G01N 23/00; G21K 1/12

(52) U.S. Cl. 378/4

(57) ABSTRACT

A method is disclosed for imaging in a medical diagnostic unit. The method includes recording a three-dimensional image data record of an examination object, reconstructing three-dimensional image data to produce a number of p images of the examination object, which respectively have a slice thickness d, assembling in each case n images to form a combined image having the slice thickness n·d, displaying at least one of the p/n combined images for the diagnosis instead of the p images, and displaying the individual produced images having the slice thickness d upon request by the user. The individual images, and no longer the combined images are automatically displayed when an actuating element of the diagnostic unit is actuated in a certain way.

### PRIORITY STATEMENT

The present application hereby claims priority under 35 U.S.C. §119 on German patent application number DE 10 2006 000 789.1 filed Jan. 4, 2006, the entire contents of which is hereby incorporated herein by reference.

### FIELD

Embodiments of the present invention generally relate to a method for imaging in a medical diagnostic unit and/or to a medical diagnostic unit therefore. Embodiments of the invention may be applied, in particular, in magnetic resonance systems or magnetic resonance tomographs (MRT), or in computed tomographs (CT).

### BACKGROUND

The development of novel imaging and recording techniques has recently rendered it possible in imaging diagnostics to record ever more images of examined persons in an acceptable recording period. For example, the use of a number of antennas by recording signals in magnetic resonance tomography has, inter alia, led to the fact that the number of phase coding steps can be reduced when recording signals. The result of this has been to produce a very large quantity of image data in a relatively short time.

The careful sifting of all the images for diagnostic purposes requires a high time outlay, however. For this reason, there is the risk that recording methods which produce an excessively large set of images are no longer used in everyday clinical practice since the inspection of the individual images would signify excessively high time outlay.

### SUMMARY

In at least one embodiment of the present invention, a method is provided for visualizing large quantities of image data that enables sifting of large image data quantities in a relatively short time.

In accordance with a first aspect of an embodiment of the invention, the latter relates to a method for imaging in a medical diagnostic unit, the method having the following steps. In accordance with one step of an embodiment of the invention, a three-dimensional image data record of an examination object is recorded. Three-dimensional image data are reconstructed after recording of this image data record in order to produce a number of p images of the examination object which respectively have a slice thickness d.

Before these individual p images are displayed, in each case n images are assembled to form a combined image having the slice thickness n·d, at least one of the now p/n combined images being displayed. Upon request by the user, however, the individual images having the slice thickness d are displayed instead of the combined images.

The method according to at least one embodiment of the invention may include the advantage that the operator or the diagnosing doctor now needs to pursue only a smaller number of images in a first overview. If, when viewing these combined images, the doctor detects anomalies that necessitate a more accurate examination, he can switch over from the display of the combined images to the display of the individual images having the small slice thickness, for example by actuating an actuating element for the diagnostic unit in a predetermined way. Upon request by the user, it is possible to zoom in the slice direction, that is to say to improve the resolution in the slice direction.

Instead of providing the individual measured images, combined images are automatically produced and displayed for diagnostic purposes, and so the overall quantity of images is reduced.

Since the inventive method of at least one embodiment can also be used to sift relatively large data records with acceptable time outlay, because the number of images to be viewed is reduced in a first overview, it is possible, in turn, to record three-dimensional image data records that have an advantage over the recording techniques in which a number of images having a specific slice thickness are recorded sequentially. For example, a three-dimensional image data record enables the production of a gapless rectangular slice profile, while the slice thickness can likewise be reduced, which reduces the partial volume effect, the result being an overall improvement in the contrast. Furthermore, the combined images have a better contrast than the corresponding measured slice with a two-dimensional imaging sequence, since a reduced dephasing in the slice direction resulted in the case of the combined images. Overall, a high signal-to-noise ratio can be achieved in the case of three-dimensional recordings.

In accordance with at least one embodiment of the invention, an isotropic three-dimensional image data record is recorded such that in the reconstructed images the pixels in the image plane have an extent that corresponds to the slice thickness. For example, the slice thickness d for each individual image can be 0.5 mm, and so the pixel resolution in the image plane is also 0.5·0.5 mm. Of course, at least one embodiment of the invention can also be used with a non-isotropic image data record, which means that the slice thickness d does not correspond to the pixel dimension in the image plane.

In accordance with a further embodiment of the invention, in each case n juxtaposed images are assembled in the production of the combined images, n lying between 2 and 12, preferably between 6 and 10, and it preferably being the case, furthermore, that n=8. Thus, given a slice thickness of 0.5 mm, the assembly of in each case eight juxtaposed slices yields a combined image having a slice thickness of 4 mm, in each case. In the event of the assembly of eight juxtaposed slices, the number of the images to be inspected is therefore reduced by the factor 8. Of course, any other number n of images can be assembled to form a combined image. The quantity of the assembled images is a function here chiefly of the size of the image data records produced, and of the clinical problem that is to be answered with the aid of the images.

In order to switch over the view of the combined images to the individual images, it is possible, for example, to actuate an actuating element of the diagnostic unit in a predetermined way. For example, one of the individual images from which the combined image is built up can be displayed by clicking the mouse on the combined image. Furthermore, when switching over from the combined image to the individual image it is possible firstly to display the individual image that is the foremost or the rearmost image in the slice direction of the combined image. It is equally possible to select an image from the middle of the combined image. If, for example, eight images are assembled to form an overall image, it is possible, for example, to display firstly the fourth or fifth image, the images lying further outside relative to the firstly displayed individual image being displayed in the event of a further inspection of these images. If the first or the last image in the slice direction is firstly displayed when switching over to the individual images, the images are displayed sequentially.

The abovenamed examples of the method according to at least one embodiment of the invention relate, for the most part, to magnetic resonance systems. Of course, embodiments of the invention can also be applied in the case of computed tomographs, where it is also possible to produce large image data records.

At least one embodiment of the invention also relates to a medical diagnostic unit that has an image data recording unit for recording a three-dimensional image data record of an examination object. The diagnostic system further has a reconstruction unit for reconstructing p images that each have a slice thickness d. The reconstruction unit continues to assemble in each case n images to form a combined image with an overall slice thickness n·d, at least one of the combined images being displayed on a display unit. Furthermore, an operating unit is provided with the aid of which the switchover is performed when required from the assembled image to the individual image, when the operating unit is actuated in a predetermined way.

### BRIEF DESCRIPTION OF THE DRAWINGS

Embodiments of the invention are explained below in more detail with reference to the attached drawings, in which:

FIG. 1 shows three-dimensional image data,

FIG. 2 shows the image data of FIG. 1 after assembly to form combined images,

FIG. 3 shows an image combined from n images, in detail, and

FIG. 4 shows a flowchart for sifting a three-dimensional data record.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

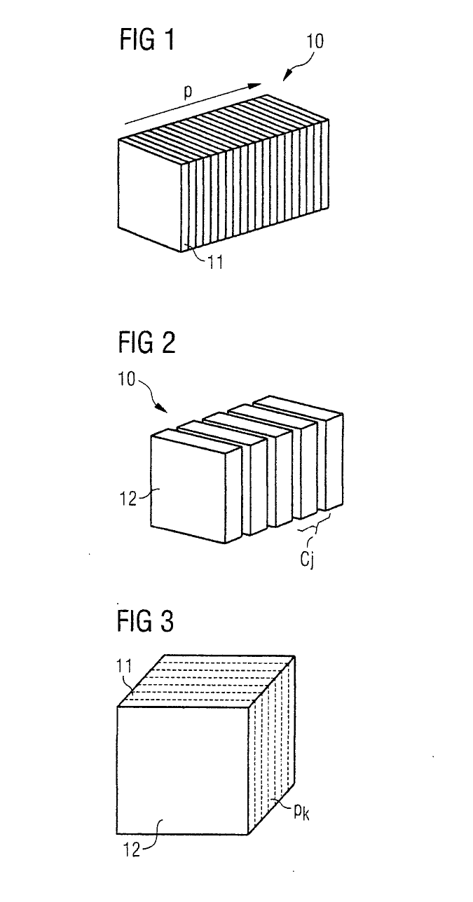

FIG. 1 shows the image data 10 reconstructed from a three-dimensional image data record, the image data 10 having p individual images 11. Each individual image 11 has a slice thickness d. The number of the individual images 11 can be, for example, 200. If the image data record is recorded as an isotropic image data record, the slice thickness of an individual image corresponds equally to the resolution of the pixels in the image plane. Such three-dimensional image data records can, for example, be used to produce multiplanar reconstructions in the case of which images can be produced at arbitrary viewing angles from the recorded examination object.

A further possibility for reprocessing three-dimensional image data is the production of maximum intensity projection (MIP) images, such as are used in the display of blood vessels. The sifting of these p individual images can signify an excessively high time outlay, and so after being recorded the images are converted into compound images that are displayed in FIG. 2.

In FIG. 2, the image data 10 are not, as in FIG. 1, displayed in individual images 11, but in each case a number of n individual images are assembled to form a combined image, the slice thickness of such an assembled image 12 being no longer d, but n·d. Owing to the fact that, from the total number of p images, n are combined in each case to form a new image, p/n combined images are yielded overall. These combined images are then displayed to the user of the diagnostic unit, who can then use these combined images for a general diagnosis.

It may be shown using the following example how the outlay for sifting the images is thereby reduced. Given that a total of p=200 individual images having a slice thickness d of 0.5 mm are recorded, and that in each case n=8 images were assembled to form a combined image, a total of 25 combined images having a slice thickness of 4 mm in each case is yielded. These 25 images can then be more quickly and more simply sifted than the original 200 images. Of course, any other number n of images can be assembled. The diagnosing doctor or the operator will select the number n as a function of the problem that he wishes to address with the aid of the imaging diagnostics.

If, when inspecting the combined images 12, the diagnostician detects an irregularity that he wishes to examine more closely, he is able to improve the resolution in the slice direction by switching over to the individual images by displaying the individual images 11. A combined image 12 is illustrated in detail in FIG. 3. This combined image 12 includes n individual images 11.

If the doctor now wishes for diagnostic purposes to switch over from the combined images 12 to the individual images 11 in the display, he is able, by actuating an operating element, to zoom in the slice direction in a predetermined way (for example by clicking the mouse) and to display the individual images 11. When switching over from displaying the combined images 12 to the individual images 11, one possibility is to proceed such that, for example, the first or the last individual image in the slice direction is firstly displayed, and it is possible to leaf through the individual images of this combined image in the slice direction.

It is likewise possible firstly to select an individual image 11 approximately from the middle of the combined image, and to be able to leaf through the individual images toward the edge starting from the middle. The combined images 12 usually have a better contrast by comparison with a two-dimensional measurement with the same slice thickness, since a reduced dephasing in the slice direction results in the case of the three-dimensional measurement.

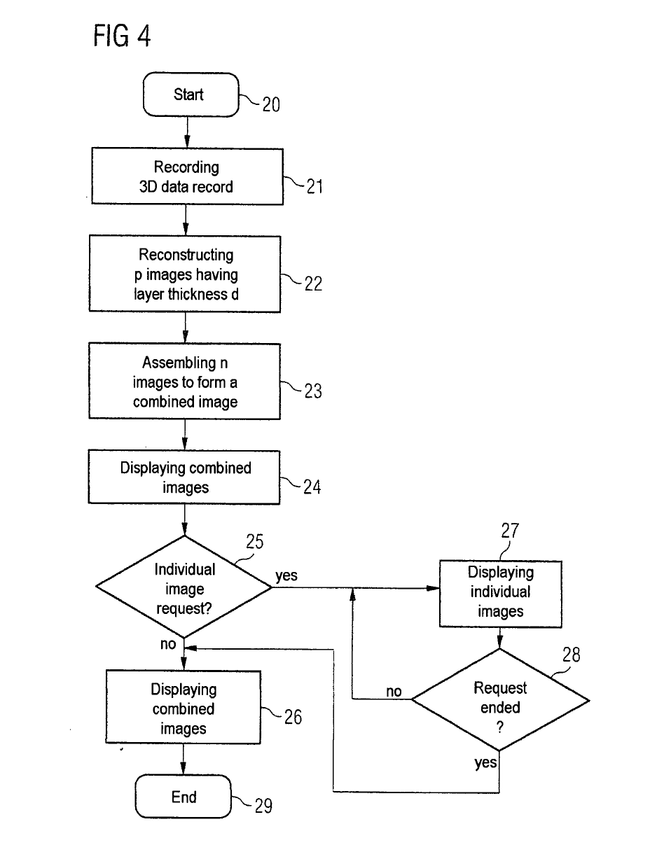

FIG. 4 illustrates by way of example the steps that can be followed by a method according to an embodiment of the invention for sifting images. After the start of the method in step 20, a three-dimensional data record of an examination object is recorded in step 21. Subsequently, p images having a layer thickness d are reconstructed from the three-dimensional data record in a step 22.

However, it is not these p images that are made available on a display unit for a general diagnosis, but in each case n sequential images of the p individual images are assembled to form a combined image in a step 23. The combined image can, for example, be calculated using the following formula:

$${C}_{j} := {\sum }_{k = j \cdot n}^{( j + 1 ) \cdot n - 1} {P}_{k} ,$$

P being the number of the 3D slices, k being a slice index that runs from 0 to P, s representing a composition factor that runs between 1<s<p and specifies how many slices are to be assembled to form a combined image. C describes the respective combined slice and j describes the slice index, where j=0 to p/n.

These combined images are displayed in a step 24 for the purpose of a general diagnosis. A check is made in a step 25 as to whether a request to switch over to individual images is present from the operator. If this is not the case, the combined images continue to be displayed (step 26).

If, however, a switchover is made to the individual images during diagnosis, the individual images are displayed in a step 27. For example, it is possible for one of the individual images to be displayed automatically instead of the combined image by pressing a mouse key. As long as, for example, the mouse key remains pressed, the individual images are displayed.

A check is made in step 28 as to whether the request to display individual images is terminated. This would be the case in the abovenamed example when the mouse key was no longer being pressed. If the mouse key continues to be pressed, the individual images are further displayed, as in step 27. If the individual image arrangement is terminated, that is to say if the pressed mouse key is released again in the above example, the combined images are displayed again as in step 26. The process ends in step 29.

Of course, any other key of a keyboard of the operating unit, or any other key combination can be selected, if appropriate in conjunction with the mouse, in order to jump back and forth between the display of the combined images and the display of individual images.

As may be gathered from the above description, embodiments of the present invention enables the use of three-dimensional image data records, it being possible for the outlay on sifting these images to be considerably reduced. If the individual images are to be displayed for the diagnosis, this can be performed by way of a simple switchover mechanism. This switchover mechanism can take place by actuating any key or any key combination of an operating element.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDS; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for imaging in a medical diagnostic unit, comprising:
recording a three-dimensional image data record of an examination object;
reconstructing three-dimensional image data to produce a number of p images of the examination object, each image including a slice thickness d;
assembling n images to form a combined image including a slice thickness n·d, and displaying at least one of the p/n combined images for the diagnosis, in place of at least one of the p images; and
displaying the individual produced images including the slice thickness d upon request by the user, the individual images, and no longer the combined images, being automatically displayed when an actuating element of the diagnostic unit is actuated.

2. The method as claimed in claim 1, wherein an isotropic three-dimensional image data record is recorded such that in the reconstructed images, the pixels in the image plane include an extent that corresponds to the slice thickness d.

3. The method as claimed in claim 1, wherein n images are assembled when preparing combined images, n being between 4 and 12.

4. The method as claimed in claim 1, wherein, when a switchover is made from displaying a combined image to displaying the individual images that form the combined image, at least one of
the foremost image or the rearmost image in the slice direction is displayed, and
the individual image that lies in the vicinity of the middle of the slice thickness of the combined image is displayed.

5. The method as claimed in claim 1, wherein the diagnostic unit is at least one of a magnetic resonance system and a computed tomography system.

6. The method as claimed in claim 1, wherein n images are assembled when preparing combined images, n being between 6 and 10.

7. The method as claimed in claim 1, wherein n images are assembled when preparing combined images, n being 8.

8. The method as claimed in claim 2, wherein n images are assembled when preparing combined images, n being between 4 and 12.

9. The method as claimed in claim 2, wherein n images are assembled when preparing combined images, n being between 6 and 10.

10. The method as claimed in claim 2, wherein n images are assembled when preparing combined images, n being 8.

11. The method as claimed in claim 2, wherein the diagnostic unit is at least one of a magnetic resonance system and a computed tomography system.

12. The method as claimed in claim 3, wherein the diagnostic unit is at least one of a magnetic resonance system and a computed tomography system.

13. The method as claimed in claim 4, wherein the diagnostic unit is at least one of a magnetic resonance system and a computed tomography system.

14. A computer readable medium including program segments for, when executed on a computer device of a magnetic resonance system, causing the magnetic resonance system to implement the method of claim 1.

15. A computer readable medium including program segments for, when executed on a computer device of a computed tomography system, causing the computed tomography system to implement the method of claim 1.

16. A medical diagnostic unit, comprising:
an image data recording unit to record a three-dimensional image data record of an examination object; and
a reconstruction unit to reconstruct p images, each including a slice thickness d, the reconstruction unit being further used to assemble n images to form a combined image with an overall slice thickness n·d, at least one of the combined images being displayed on a display unit; and
an operating unit to perform a switchover from the assembled image to an individual image, when actuated.

17. The medical diagnostic unit as claimed in claim 16, wherein an isotropic three-dimensional image data record is recorded such that in the reconstructed images, the pixels in the image plane include an extent that corresponds to the slice thickness d.

18. The medical diagnostic unit as claimed in claim 16, wherein, when a switchover is made from displaying a combined image to displaying the individual images that form the combined image, at least one of
the foremost image or the rearmost image in the slice direction is displayed, and
the individual image that lies in the vicinity of the middle of the slice thickness of the combined image is displayed.

19. A medical diagnostic unit, comprising:
means for recording a three-dimensional image data record of an examination object;
means for reconstructing three-dimensional image data to produce a number of p images of the examination object, each image including a slice thickness d;
means for assembling n images to form a combined image including a slice thickness n·d, and displaying at least one of the p/n combined images for the diagnosis, in place of at least one of the p images; and
means for displaying the individual produced images including the slice thickness d upon request by the user, the individual images, and no longer the combined images, being automatically displayed when an actuating element of the diagnostic unit is actuated.

20. The medical diagnostic unit as claimed in claim 19, wherein, when a switchover is made from displaying a combined image to displaying the individual images that form the combined image, at least one of
the foremost image or the rearmost image in the slice direction is displayed, and
the individual image that lies in the vicinity of the middle of the slice thickness of the combined image is displayed.
